(19) United States
(12) Patent Application Publication
(10) Pub. No.: US 2026/0286385 A1
(43) Pub. Date: Sep. 24, 2026

(54) HIGH EUKARYOTIC CELL DENSITY TRANSIENT TRANSFECTION PROCESS FOR MANUFACTURING OF RECOMBINANT VIRAL VECTORS

(71) Applicant: FIXELL BIOTECH AB, Stockholm (SE)

(72) Inventors: Veronique CHOTTEAU, Nacka (SE); Ye ZHANG, Taby (SE); Emil SUNDANG PETERS, Sollentuna (SE)

(21) Appl. No.: 18/877,616

(22) PCT Filed: Jun. 22, 2023
(86) PCT No.: PCT/SE2023/067049

§ 371 Date: Dec. 20, 2024

(30) Foreign Application Priority Data
Jun. 24, 2022 (SE) 2230210-3

(51) Int. Cl. C12N 15/86

(52) U.S. Cl. CPC C12N 15/86; C12N 2750/14143; C12N 2750/14151

(57) ABSTRACT

The present disclosure provides a process for producing recombinant viral vectors in eukaryotic cells. In particular, the present disclosure relates to a process for the transient transfection of eukaryotic cells for production of recombinant viral vectors comprising a step of transient transfection of cells in a cell culture, wherein the cell culture has a surprisingly high cell density of at least 25 MVC/mL. After the transfection step, an optional cell recovery step can be performed depending on the host cell type, the exogenous nucleic acid(s) and the transient transfection method that are used. After the transfection step or after an optional cell recovery step, the production step of recombinant viral vectors starts at a surprisingly high cell density of at least 20 MVC/mL, with limited dilution or no dilution performed after the transfection step.

### FIELD OF THE INVENTION

The present disclosure relates to the field of transient transfection of cells and the production of recombinant viral vectors. More specifically, the disclosure relates to a process for high cell density transient transfection for the production of recombinant viral vectors.

### BACKGROUND TO THE INVENTION

Transient transfection is a gene transfer technology that is widely used to produce biologics, which are molecules or molecule assemblies with a biological activity, to introduce exogenous nucleic acid(s) into host cells for a limited period of time instead of integrating into the host cell genome. After the transient transfection the cells that have incorporated the target nucleic acid(s) are able to express the transfected genetic materials and enter the production phase of the biologic, which can be proteins, polymers, virus, virus-like particles, extra-cellular vesicles, etc. In contrast to using stable producer cell lines which takes up to years to establish, transient transfection requires only a few days and is extremely versatile to host cell lines, transfection vectors, as well as target products. These advantages make it a superb candidate for the manufacturing of large molecules such as recombinant proteins and viral vectors, during drug discovery studies and pre-clinical phase where intensive screenings and characterization of a target product and its manufacturing process parameters are performed, or in case the expressed modality includes a toxic part. However, biopharmaceutical industry has been constantly reported lacking productivities for molecules such as viral vectors, due to bottlenecks in the manufacturing process such as low production yield and high production cost.

In a transient transfection, plasmid DNA is one of the common genetic materials that is delivered into the host cells. Plasmid DNA is a small, circular, double-stranded DNA molecule and in some cases a co-transfection of multiple plasmids is needed to produce certain products. For example, for the recombinant Adeno-Associated Viral vectors (rAAV) production in the gene and cell therapy fields, multi-plasmid transient transfection of HEK293 (Human Embryonic Kidney 293) is currently the most widely used method [Xiao, et al. “Production of high-titer recombinant adeno-associated virus vectors in the absence of helper adenovirus.” J Virol 72 (1998): 2224-2232; Grimm, et al. “Novel tools for production and purification of recombinant adeno associated virus vectors.” Hum Gene Ther9 (1998): 2745-2760; Matsushita, et al. “Adeno-associated virus vectors can be efficiently produced without helper virus.” Gene Ther 5 (1998): 938-945]. The co-transfection is usually more challenging as all genetic elements must be delivered simultaneously, but in some cases remains the only or preferred option due to technical restrictions. Furthermore, the biosynthesis of recombinant viruses represents a very high level of complexity, as a matter of fact, it implies the biosynthesis and correct assembly of a capsid made of a total of 60 molecules of viral proteins, as well as the inclusion of the genome of interest in the capsid. It is well known that this manufacturing process becomes less efficient when the cell density is increased on a cell specific basis, so called cell density effect, [Maranga, L., et al. 2005. “Characterization of changes in PER. C6™ cellular metabolism during growth and propagation of a replication-deficient adenovirus vector.” Biotechnology and bioengineering, 90(5), pp. 645-655; Henry, O. et al. 2005. “Metabolic flux analysis of HEK-293 cells in perfusion cultures for the production of adenoviral vectors.” Metabolic engineering, 7(5-6), pp. 467-476]. Similar observations have been reported for the production of rAAV by insect cells using the baculovirus-based expression system [Joshi et al., 2021. “Advancements in molecular design and bioprocessing of recombinant adeno-associated virus gene delivery vectors using the insect-cell baculovirus expression platform”. Biotechnology Journal, 16(4), p. 2000021; Meghrous et al., 2005. “Production of recombinant adeno-associated viral vectors using a baculovirus/insect cell suspension culture system: From shake flasks to a 20-L bioreactor”. Biotechnology progress, 21(1), pp. 154-160; Urabe et al., 2002. “Insect cells as a factory to produce adeno-associated virus type 2 vectors”. Human gene therapy, 13(16), pp. 1935-1943].

The recombinant viral vectors production process is typically divided into 3 steps: first step to achieve a desired number of cells, i.e., by cell expansion, transient transfection step and production step. Cells that proliferate in culture vessel(s) are first prepared for the transient transfection, which is typically done by setting the density of growing cells at around 0.6 to 0.8 million Viable Cells per milliliter (MVC/mL) one day before transfection. At the day of transfection, the cell density achieves around 1 to 1.5 MVC/mL from cell growth, preferably in exponential growth. This operation provides cells hosts for the transfection with high cell viability and in culture conditions favorable for the transfection efficiency.

In the case of transfections performed at high cell density, a high cell density cell suspension can be obtained by concentration of low cell density cell cultures, or by culturing the cells at high density. After the transient transfection the cells that have incorporated the target nucleic acid(s) are able to express the transfected genetic materials and enter the production phase. In the case of high cell density transfections, a dilution step post transfection is usually performed to set the cells back to low cell density cultures to enable the production. The time point to perform a dilution and the resulting cell density after dilution are highly dependent on the host cell type, the transient transfection method that is used, laboratory instrument and control systems that are available. After a certain time period of production, typically 48 to 72 hours post transfection (hpT), the produced recombinant viral vectors are harvested and the production titers are well examined before proceeding to next step of manufacturing, i.e., purification etc.

There are various non-viral transient transfection methods commercially available including physical and chemical methods. Chemical transfection is a widely adopted method that utilizes a transfection reagent to form a complex with target nucleic acids and the target nucleic acids are then delivered through the cellular membrane to host cells via the formed complex. Among the wide variety of transfection reagents that are commercially available, Polyethylenimine (PEI) is a stable cationic polymer that condenses the negatively charged DNA into PEI-DNA complex particulates. The complex particulates then bind to the cell surfaces and get transferred into the cells via endocytosis/phagocytosis. PEI-mediated transfection process is a popular technique due to its relatively low cost and ease of operation. According to the state-of-the art technologies, chemical transfections for molecules productions are prevalently performed at cell densities≤5 MVC/mL with the standardized protocols operated at 1-2 MVC/mL.

Another popular physical transfection technique is electroporation, where the transient transfection process can be done at high cell densities (up to 100 MVC/mL). This is done by concentrating the cell broth to a very high target cell density, usually via centrifugation, right before the electroporation. After the electroporation the cell suspension is diluted back to low cell densities such as ≤5 MVC/mL, such as 1-2 MVC/mL, to enter the production phase. The application of this technique is however limited by the operational scale and cost. Electroporation is used in lab scale that is per batch below 6 mL, commonly at the scale of below 1 mL. Solution towards larger scale exist such as flow electroporation technology commercially provided by the company Maxcyte that can process up to 100 mL cell suspension at 100 MVC/mL per batch, but a dilution back to low cell densities such as ≤5 MVC/mL, such as 1-2 MVC/mL, for production phase is necessary within 1 hour after the electroporation.

Accordingly, the prior art methods suffer from a number of drawbacks, such as low production yields, restriction to small operational scale and high production costs. An objective of the present invention is to obviate these drawbacks and provide a process for transient transfection of cells and production of recombinant viral vectors with potential for use in large scale to lower costs.

### SUMMARY OF THE INVENTION

The present disclosure provides a process or method for producing recombinant viral vectors in eukaryotic cells. Herein, a process may also be referred to as a method, i.e., said terms are used interchangeably herein.

In particular, the present disclosure relates to a process for the transient transfection of eukaryotic cells for production of recombinant viral vectors comprising a step of transient transfection of cells in a cell culture, wherein the cell culture has a surprisingly high cell density of at least 25 MVC/mL. After the transfection step, an optional cell recovery step or process can be performed depending on, or according to, the host cell type, the exogenous nucleic acid(s), or the exogenous plasmid DNA(s) and the transient transfection method that are used. After the transfection step or after an optional cell recovery step or time period, the production step of recombinant viral vectors starts at a surprisingly high cell density of at least 20 MVC/mL, with limited dilution or no dilution performed after the transfection step.

The present method comprises thus:
a) obtaining a cell culture having a cell density of at least 25 million viable cells per milliliter;
b) performing a transient transfection of the cells in the cell suspension with an exogenous plasmid DNA or several exogenous plasmid DNAs that are needed to produce the recombinant viral vector, i.e., a co-transfection with multi-plasmid DNA;
c) optionally a cell recovery for a period of time depending on the host cell type, the exogenous plasmid DNA(s) and the transient transfection method that is used. During the cell recovery period, the said transfected cells are kept in a culture or solution with a large surface beneficial for gas exchange such as oxygen or air exchange, usually the transfected cells are kept still, or essentially still and are usually kept still on a large enough surface. This time period is usually larger than 1 minute and varies depending on the host cell type, the exogenous plasmid DNA(s) and the transient transfection method that is used;
d) Following the transfection step b), and the optional cell recovery step c), the production step is initiated after limited or no dilution to said transfected cells to begin the production phase from a cell density of at least 20 million viable cells per milliliter following the transfection step.

In other words, it is also disclosed herein a process for producing recombinant vectors in eukaryotic cells, said method comprising the steps of:
a) obtaining a cell suspension having a cell density of at least 25 million viable cells per milliliter;
b) performing a transient transfection of the cells in the cell suspension with an exogenous plasmid DNA or a plurality of different exogenous plasmid DNAs to obtain transfected cells;
c) optionally performing a cell recovery for a period of time, wherein during the cell recovery period, said transfected cells are kept essentially still on a surface; and
d) following the transfection of step b), and/or the optional cell recovery step c), limited or no dilution is performed to said transfected cells present in said cell suspension and wherein production of said recombinant vectors is thereafter initiated from a culture of transfected cells having a cell density of at least 20 million viable cells per milliliter.

The surface of step c) mentioned above is usually a surface of a container or a vessel or the like, as described elsewhere herein.

The target recombinant viral vector is then harvested some time post transfection, typically 24-240 hours post transfection (hpT), but longer production times are also envisaged, as disclosed elsewhere herein.

By partially or completely eliminating the dilution step back to low cell density such as ≤5 MVC/mL, such as to 1-2 MVC/mL after transfection, the manufacturing process is largely simplified. The same vessel can be used for cell cultivation, transfection and production, from the starting host cell inoculation to the final harvest of the target recombinant viral vector. The manufacturing footprint is therefore minimized to a great extent and makes the whole manufacturing process scalable to large-scale industrial production.

### BRIEF DESCRIPTION OF THE DRAWINGS

FIG. 1 illustrates the titers of rAAV capsids/mL produced when different ratios of cell number to plasmid DNA, and plasmid DNA to PEI (Polyethyleneimide) were used in a transient transfection process of the present invention performed at 50 MVC/mL. Empty bars: controls, with cell density at transfection 1 MVC/mL, in two different media; Filled bars: 3^{rd} to 12^{th} groups with cell density at transfection 50 MVC/mL with ratios between cell number (million cells), plasmid DNA (μg) and PEI (μg). In the groups 1:1:1, 1:1:2, 1:1:4 and 1:1:8, the ratio of plasmid DNA to cell number is 1 μg DNA per 1 MVC, with DNA (μg): PEI (μg) ratios 1:1, 1:2, 1:4 and 1:8 respectively. In the groups 1:2:2, 1:2:4 and 1:2:8, the plasmid DNA is 2 μg DNA per 1 million cells, with DNA (μg): PEI (μg) ratios 1:1, 1:2 and 1:4 respectively. In the groups 1:4:4, 1:4:8 and 1:4:16, the plasmid DNA is 4 μg DNA per 1 million cells, with DNA (μg): PEI (μg) ratios 1:1, 1:2 and 1:4 respectively.

FIG. 2 illustrates the production titers of rAAV capsids/mL at 72 hpT for a transient transfection process performed at 50 MVC/mL with different incubation times of the cationic polymer and DNA, i.e. 5 mins, 10 mins, 15 mins or 0 min (‘Quick mix’), as well as DNA added into the cell suspension, followed by immediate addition of cationic polymer into the cell suspension without pre-mixing of cationic polymer and DNA (‘Direct transfection’).

FIG. 3 illustrates the production titers of rAAV capsids/mL after a transient transfection process of the present invention performed at 100 MVC/mL, followed by dilution at different cell densities for the AAV production harvested at 72 hpT, using different medium exchange frequencies; from left to right:
1) dilution to 10 MVC/mL, with medium exchange at 24 hpT and 48 hpT, as control to diluted cell density outside of the present invention;
2) dilution to 10 MVC/mL, with one medium exchange at 48 hpT, as control to diluted cell density outside of the present invention;
3) dilution to 30 MVC/mL, with medium exchange at 18 hpT, 42 hpT and 66 hpT;
4) dilution to 30 MVC/mL, with medium exchange at 24 hpT and 48 hpT.

FIG. 4 illustrates the production of rAAV viral vector via the transient transfection process of the present invention at cell density 64 MVC/mL in a perfusion bioreactor culture with 220 mL working volume. Filled circles: viable cell density (MVC/mL); empty circles: viability (%); empty squares: transfection efficiency (GFP signal %).

FIG. 5 illustrates the production of rAAV viral vectors via a transient transfection process of the present invention using a flow electroporation at cell density >80 MVC/mL in a perfusion bioreactor culture with 200 mL working volume. Filled circles: viable cell density (MVC/mL); empty circles: viability (%); empty squares: transfection efficiency (GFP signal %).

FIG. 6 illustrates the production of rAAV viral vectors performed thanks to a transient transfection process of the present invention at 50 MVC/mL in a perfusion bioreactor culture with 200 mL working volume, showing as well two control cultures of 2 MVC/mL viable cell density and of 15 MVC/mL viable cell density where the transfection process was performed in pseudo-perfusion mini bioreactors, i.e., volume 50 mL vessels with vent caps, in a 37° C. CO₂ incubator.

FIG. 6A shows a long-term production in bioreactor with viable cell density up to 120 MVC/mL during the production phase; filled circle with solid line: viable cell density; empty circle: cell viability; empty square with dotted line: transfection efficiency as GFP expression; empty diamond: production titer viral genome/mL; Control cultures in mini bioreactor with transfection performed at 1 MVC/mL cell density (left pattern-filled circle) with viral genome production/mL (left filled diamond) and Control culture in mini bioreactor with transfection performed at 15 MVC/mL cell density (right pattern-filled circle) with viral genome production/mL (right filled diamond) represented in the same graph for convenience but the time scale does not apply for these.

FIG. 6B shows long-term consistent high infectious titers, which demonstrates the biological efficiency of the produced viral vectors, of supernatant samples collected from the bioreactor run of FIG. 6A at 3 days, 6 days and 7 days post transfection (3 dpT, 6 dpT and 7 dpT), used for a transduction assay demonstrated by the GFP expression of the infected cells (filled bars). For comparison a Reference (Ref) in which the transfection is done in a shake flask at low cell density of 1 MVC/mL and the supernatant is collected 3 days post transfection, which is the common practice for rAAV production.

FIG. 7 illustrates several transient transfection production processes of rAAV viral vectors performed with the present invention at cell density 50 MVC/mL. Two cell lines were used: HEK293F and Viral production cells 2.0 (VPC2.0). Two different chemical transfection methods, cationic polymer PEI and cationic lipid-based transfection reagent (VPT), were used and three transfection media, Viral production medium (VPM), FreeStyle293 (FS293) and BalanCD HEK (BCD), were used. The cell densities and viabilities at harvest 3 dpT, together with transduction assay performed using supernatant from harvest 3 dpT are shown in FIG. 7A, and the associated production titers are showed in FIG. 7B.

### DETAILED DESCRIPTION OF THE INVENTION

### Definitions & Abbreviations

Terms not specifically defined herein should be given the meanings that would be given to them by one of skill in the art in light of the disclosure and the context. As used in the specification unless specified to the contrary, the following terms have the meaning indicated.

The term “transient transfection” as used herein means that the introduced nuclei acid is present in the cell potentially only for a limited period of time and that it is not necessarily integrated into the cell genome. The transfected cells then express the transiently transfected gene potentially for a finite period of time, which is usually limited to several days. A usual model for transfections is to express the green fluorescence protein, GFP, as gene of interest. The production of GFP can be easily quantified based on its fluorescence in the cells successfully transfected. The “transfection efficiency” gives the percentage of cells expressing GFP among all the cells. In the current disclosure, a gene encoding for GFP is used as gene of interest or cargo to be delivered by the AAV used as gene therapy.

The term “cell suspension” as used herein refers to a solution of cells not adhering to a static support. Such cells typically grow as culture in stirred tank bioreactor or agitated culture vessels in a mixture ensuring nutrient delivery to the cells. A cell suspension could be obtained by cells growing in culture, by a concentration procedure and/or by a dilution procedure. Herein, this term is used interchangeably with the terms or expressions “cell culture suspension”, “suspension comprising cells”, or the like.

The term “cell density” as used herein means the concentration of cells in a given volume and quantifies the biomass in terms of cell concentration. Accordingly sometimes herein the term “cell density” is used interchangeably with the expression “concentration of cells”, or the like. The often-used unit is million viable cells per milliliter (MVC/mL) and it is widely used for eukaryotic cells. The quantification of the cells can also be expressed as cell volume fraction; for example, in the current disclosure, in a cell suspension of cell density 25 MVC/mL with an average cell diameter of 17-22 μm, the cells occupy at least a fraction of 6 percent of the culture volume (volume/volume).

Herein, when a “limited or no dilution is performed” as disclosed herein, this means that the dilution is limited in the sense that the starting cell density for production of said recombinant vectors is at least 20 million viable cells per milliliter, compared to conventional methods where a dilution to low cell density≤5 MVC/mL, typically 1-2 MVC/mL, is commonly performed to initiate the production of said recombinant vectors.

The term “perfusion” as used herein, is a process in which the cells are retained inside a bioreactor or a culture vessel using a dedicated device connected to the bioreactor or culture vessel while constantly exchanging the medium in said reactor or vessel, at points in time when this is considered suitable for the specific process. The medium exchange provides cells with fresh culture medium while removing spent medium containing cellular waste and byproducts. This process is known in the art, and so are also any variants of this process that may be applied by the skilled person to the present disclosure.

The term “pseudo-perfusion” as used herein, is a process in which the purpose is comparable to the perfusion process, i.e., the spent medium is completely or partially removed and replaced by fresh medium at points in time and proportion suitable for the specific process, but the operation is performed by centrifugation or sedimentation of the cells. This procedure can be performed manually or automatized using a robot handler, or variants of these.

A “perfusion bioreactor culture” as referred to herein, is a cell culture run in perfusion process in a bioreactor according to the present disclosure.

Riedl et al. has previously reported a non-viral transfection of Human T Lymphocytes where the transfection process is performed up to 40 MVC/mL [Riedl, et al. “Non-Viral Transfection of Human T Lymphocytes.” Processes 2018, 6, 188]. This number should not be considered directly as such, since naïve T cells have a diameter of approximately 5-7 μm. In case Human T Lymphocytes cells would occupy a fraction of 6 percent of the culture volume, the cell density of these cells would be 530 MVC/mL, while 6 percent of a culture volume of cells with 17-22 μm diameter corresponds to a density of 25 MVC/mL as described in the current disclosure. The term “Cell recovery” as used herein means an optional time period post transfection, to allow the cell recovery from the transfection process to improve the cell viability and further facilitate the production of targeted product of interest. In a non-viral transient transfection gene delivery, no matter what method one uses, the foreign DNA must overcome several barriers to successfully express the target gene it carries. This process involves the host cell membrane being disrupted to a certain extent to allow the introduction of foreign DNA into the cells, followed by trafficking the DNA toward the nuclear envelope inside cytoplasmic compartment, and finally cross the nuclear envelop. After that, gene expression is only produced when enough intact DNA get into the cell nucleus. This is also why all the transfection methods are considered partly deleterious to the cells.

A cell recovery post transfection is sometimes adopted for non-viral gene delivery methods such as electroporation as cell viability decreases a lot during the transfection. This is however not commonly used in transfection methods using a chemical reagent since the cell density adopted in chemical transfection is generally very low, ≤5 MVC/mL, typically 1 or 2 MVC/mL, so that the cells are not subject to stress such as limited access to nutrients and oxygen, which normally happens in cell suspension with high cell densities.

In the current disclosure, the transient transfection process is based on high cell densities, therefore the optional cell recovery process is adopted in two of the embodiments. In the two embodiments where the cell recovery process is applied post transfection, the transfected cell suspension at high cell densities is transferred into large vessels with large surface area where the transfected cell suspension is well spread out to gain access to sterile air/oxygen. The cell recovery is not a necessary step in the current disclosure and the cell recovery time can vary over a large range, depending on the transfection method used, the cell type, plasmid DNA, etc.

Embodiment 4 herein is an example for the AAV production without cell recovery process.

In addition to the definitions above, the following abbreviations are used in the description and exemplary embodiments. If an abbreviation used herein is not defined, it has its generally accepted meaning.

| | |
|---|---|
| AAV | Adeno Associated Virus |
| rAAV | recombinant Adeno Associated Viral vector |
| hpT | hours post Transfection |
| dpT | days post Transfection |
| MVC | Million Viable Cells |
| PEI | Polyethylenimine |
| GFP | Green Fluorescent Protein |

### DETAILED DESCRIPTION

The present disclosure provides processes for high cell density transient transfection of eukaryotic cells to produce recombinant viral vectors in high yield. Such processes are for use in large scale production of recombinant vectors to streamline and lower the costs commonly associated with such processes. Specific process steps or circumstances discussed in the context of any method or process herein is equally applicable to any other process or method disclosed herein.

A method or process as disclosed herein is an in vitro method or process and cells for use in any such method or process have been isolated from their natural environment. Accordingly, cells that have been “obtained” or “provided” in a method as disclosed herein or the like are not directly obtained from their natural environment.

Herein, more specifically, there is provided a process for producing recombinant vectors in eukaryotic cells, said method comprising the steps of: a) obtaining a cell suspension having a cell density of at least 25 million viable cells per milliliter; b) performing a transient transfection of the cells in the cell suspension with an exogenous plasmid DNA or a plurality of different exogenous plasmid DNAs to obtain transfected cells; c) optionally performing a cell recovery for a period of time, wherein during the cell recovery period, said transfected cells are kept essentially still on a surface; and d) following the transfection of step b), and/or the optional cell recovery step c), limited or no dilution is performed of said transfected cells present in said cell suspension and wherein production of said recombinant vectors is thereafter initiated from culture of transfected cells having a cell density of at least 20 million viable cells per milliliter.

The “production” may sometimes herein also be referred to as the “production phase” and relates to the production of the recombinant vectors. The surface is preferably a surface in a container or a vessel, such as that large exchange of oxygen is provided.

There is also provided a process wherein said cell suspension of step a) has a cell density or concentration of at least 30 million cells per milliliter. There is also provided a process wherein said cell suspension of step a) has a cell density or concentration of at least 50 million cells per milliliter. There is also provided a process wherein said cell suspension of step a) has a cell density or concentration of at least 80 million cells per milliliter. There is also provided a process wherein said cell suspension of step a) has a cell density or concentration of at least 100 million cells per milliliter. Naturally, the production of step d) that is initiated after the transfection of step b) or step c) may, when the cell suspension of step a) has a higher cell density or concentration, such as 80 million cells per milliliter, be initiated from a cell density or concentration that is close to 80, such as 75 or 78 million cells per milliliter.

A similar reasoning is applicable to the other examples of cell densities or cell concentrations disclosed herein, i.e., the initial cell density or concentration of step a) as compared to the density or concentration of step d). As noted, the terms cell density and cell concentration may be used interchangeably herein.

There is also provided a process wherein said process comprises medium exchange performed at one or more points in time when the production of step d) has been initiated. Such a medium exchange can be performed at different time points, such as after 0, 2, 4, 6, 8, 10, 12, 14, 16, 18, 20, 22, 24 and/or after 48 hours, but not limited to, after initiation of the production. There is also provided a process comprising perfusion.

There is also provided a process, wherein said process comprises medium exchange carried out by perfusion when the production or production phase of step d) has been initiated. Hence the medium exchange is performed during the production of step d). A perfusion may be performed in a perfusion bioreactor culture, as illustrated herein.

Herein, it is beneficial, as explained elsewhere herein, that no, or limited or partial, dilution is performed before the initiation of the production of step d).

There is also provided a process wherein no dilution that would generate a cell density lower than 20 MVC/mL is performed before the initiation of the production of step d).

The medium exchange and perfusion process was shown to be of particular relevance for maintaining a high cell density after the transfection process and for obtaining a high production yield of recombinant vectors after finalizing the production phase.

Alternatively to perfusion bioreactor culture, a pseudo-perfusion process can be used.

The production of step d) of a process as described herein may be performed for a period of at least 24 hours, such as at least 48 hours or 72 hours, or for 4, 5, 6, 7, 8, 9 or 10 days. As shown in the examples, the yield of the recombinant vectors may benefit from longer production periods.

The cell suspension of step a) may be such that the cells of said suspension occupy at least a fraction of 6 percent of a culture volume and wherein the cell suspension is selected from the group consisting of a cell culture, a microorganism fermentation, a cell suspension derived from cell tissue and a cell suspension derived from blood.

Herein, the eukaryotic cells may be selected from the group consisting of mammalian cells, human cells, avian cells, insect cells and plant cells. More specifically, the cells may be selected from the group consisting of: CHO, CHO-DBX11, CHO-DG44, CHO-S, CHO-K1, Vero, BHK, HeLa, COS, MDCK, HEK-293, HEK-293T, HEK-293S, HEK-293F, L293, NIH-3T3, W138, BT483, Hs578T, HTB2, BT20, T47D, NS0, CRL7030, HsS78Bst cells, PER.C6, SP2, SP0, hybridoma, MRC-5, MDCK, WI-98, CAP, EB66, HUVEC, AGE1.CR, CR, cells of Trichoplusia ni, cells of Spodoptera Frugiperda, SF9, SF21, Hi5, mesenchymal stem cells, endothelial cells, induced pluripotent stem cells, primary cells, cells of Nicotiana tabacum, BY2, cells of Nicotiana benthamiana, cells of Oriza sativa, cells of Arabidopis thaliana, and cells of Daucus carota.

Cells for use in the present disclosure do not include cells that involve the destruction of a human embryo. Accordingly, such cells are disclaimed from and do not form part of the present disclosure.

Herein, the recombinant viral vector may be a viral vector, a virus, viral-like particles (VLPs) or any combination of these, or assemblies derived from these, or assemblies derived from these and associated with a small molecule of a size less than 1000 kDa. More specifically herein recombinant Adeno-Associated Virus (AAV) viral vector particles may be produced, optionally wherein the transfection of step b) of a process is performed using an (AAV) transient transfection system comprising co-transfection of at least three plasmids. This is exemplified in the experimental section and this system is well known to a person skilled in the art.

The cell density of step a) of the process may be obtained by adjusting the cell density of the culture suspension by concentrating the content of the cells of the cell suspension and/or by cultivation of the cells in the culture suspension. Alternatively, the cell density of step a) may be adjusted by concentrating the cells of the suspension such as by centrifugation, filtration, microfiltration, sedimentation, acoustic settling, or specific binding of the cells, or by diluting the cells of the suspension. Furthermore, the cell density of step a) of the process may be adjusted by cultivation and the cultivation is such as batch cultivation, fed-batch cultivation, perfusion cultivation, chemostat, or combination of at least two of these cultivation modes, and wherein cultivation may be performed in combination with cell concentration and/or in combination with cell dilution. These are all procedures well known in the art.

Herein, said transient transfection may comprise chemical transfections utilizing chemical reagent selected from the group consisting of: cationic polymers such as DEAE-dextran, polybrene, polyethylenimine and derivatives thereof, dendrimers, calcium phosphate, polycations; cationic lipids including liposomal and non-liposomal transfection reagents and lipid nanoparticles. Said polyethylenimine may be a synthesized cationic polymer with topologies of linear or branched forms, with a molecular weight ranging from 1 kDa to 1,000 kDa. Further herein, said transfection may comprise physical transfection methods from the group consisting of: microinjection, optical transfection, biolistic transfection (also known as particle bombardment), electroporation, iron oxide nanoparticle delivery (also known as magnetofection), sonoporation, laser irradiation, electric field-induced molecular vibration.

The present disclosure also provides a method for, more specifically, producing AAV viral vectors by eukaryotic cells. In particular, the present disclosure relates to a process for the transient transfection of eukaryotic cells for production of AAV viral vectors comprising a step of transient transfection of cells in a cell suspension, wherein the cell suspension has a cell density of at least 25 MVC/mL. After the transfection an optional cell recovery process could be performed according to the host cell type, the plasmid DNA and the transient transfection method that is used. After the transfection or the optional cell recovery time period, the production of AAV viral vectors starts at a cell density of at least 20 MVC/mL, with limited or no dilution step performed post transfection. High cell density is one of the key factors in one bioproduction process to generate high expression levels of target products. Very high cell density can significantly increase the volumetric yield of produced product of interest. However, to date a very high cell density transfection followed by very high cell density production has not been reported for the production of viral vectors by transient transfection process. This is due to a limitation linked to the cell density and to the cell fragility at transfection. It is challenging to maintaining a culture at high cell densities while the cells are still producing, especially at higher than 50 MVC/mL, while keeping a good viability in average. These are the reasons why transient production of viral vectors are not performed in this range of cell densities. As mentioned before, all the transfection methods are deleterious to the cells.

The key factor to achieve a high cell density culture is an average high viability, which is very difficult to maintain in a transient transfection process. Due to the largely increased cell number that need to be transfected in a high cell density culture, the quantity of plasmid DNA and in the cases of chemical transfections, chemical transfection reagents, are largely increased as well, which raises toxicity concern to the cells. This will consequently compromise the cell viability and lead to low yield of viral vectors.

Notice as well that a dilution step to low density after high cell density transfection is not efficient from a process point of view and considered as a disadvantage. It poses obstacles to streamline processing as well as increases medium usage, which further lead to a much bigger footprint of the whole manufacturing and raises unwanted storage and transportation issues. Prior to current disclosure, Backliwal et al. have reported in situ transfection of suspension-adapted HEK-293 cells with 25-kDa linear polyethyleneimine at densities up to 20 MVC/mL in complex media followed by production at lower cell densities (1 MVC/mL) [Backliwal et al. “High-density transfection with HEK-293 cells allows doubling of transient titers and removes need for a priori DNA complex formation with PEI.” Biotechnol Bioeng. 2008 Feb. 15; 99(3):721-7]. Blackstock et al. have also reported a transfection process to produce virus-like particles at higher transfection cell densities of 15-25 MVC/mL [Blackstock et al. “Comprehensive Flow Cytometry Analysis of PEI-Based Transfections for Virus-Like Particle Production”, Research, 2020, Article ID 1387402]. In their work, the cells are centrifuged and cell density is set to 1 MVC/mL three hours post transfection, which means the transfected cell suspension is diluted to 1 MVC/mL where the production phase starts. The dilution step after the transfection is so far necessary for a high cell density transfection process. Dilution is especially mentioned when using physical transfection method such as electroporation. In an electroporation method, high cell densities have the advantage of physical close distance between the host cells and the plasmid DNA, but the cells need to be diluted post-electroporation to start the production phase. So far, the highest reported cell density post-electroporation is 10 MVC/mL [Steger et al. CHO-S antibody titers >1 gram/liter using flow electroporation-mediated transient gene expression followed by rapid migration to high-yield stable cell lines. J Biomol Screen. 2015 April; 20 (4):545-51].

The present disclosure provides a method that comprises a step of transient transfection of cells in a cell suspension, wherein the cell suspension has a cell density of at least 25 MVC/mL. After the transfection an optional cell recovery process could be performed if needed. After the transfection or the optional cell recovery time period, the production of viral vectors starts at a cell density of at least 20 MVC/mL, with limited or no dilution step performed post transfection.

In other words, the present disclosure provides a method or a process, wherein said process comprises:
a) obtaining a cell suspension having a cell density of at least 25 million viable cells per milliliter;
b) perform a transient transfection of the cells in the cell suspension with an exogenous plasmid DNA or several exogenous plasmid DNAs that are needed to produce the recombinant viral vector, i.e. a co-transfection with multi-plasmid DNA;
c) optionally a cell recovery for a period of time, depending on the host cell type, the exogenous plasmid DNA(s) and the transient transfection method that is used. During the cell recovery period, the said transfected cells are kept still on a large enough surface. This time period can vary very much depending on the host cell type, the exogenous plasmid DNA(s) and the transient transfection method that is used;
d) Following the transfection step b, and/or the optional cell recovery step c, limited or no dilution to said transfected cells to begin the production phase from a cell density of at least 20 million viable cells per milliliter.

In some embodiments of the process of this disclosure, the cell culture suspension has a cell density of at least 30 million cells per milliliter.

In some embodiments of the process of this disclosure, the cell culture suspension comprising cells has a cell density of at least 50 million cells per milliliter.

In some embodiments of the process of this disclosure, the cell culture suspension has a cell density of at least 80 million cells per milliliter.

In some embodiments of the process of this disclosure, the cell culture suspension has a cell density of at least 100 million cells per milliliter.

In some embodiments of the process of this disclosure the cell culture suspension is such that the cells occupy at least a fraction of 6 percent of the culture volume and the cell culture suspension is selected from the group consisting of cell culture, microorganism fermentation, cell suspension derived from cell tissue and cell suspension derived from blood.

Cells useful for the process of this disclosure are typically eukaryotic cells, such as mammalian cells, human cells, avian cells, insect cells and plant cells.

Typically, the cells are selected from the group consisting of: CHO, CHO-DBX11, CHO-DG44, CHO-S, CHO-K1, Vero, BHK, HeLa, COS, MDCK, HEK-293, HEK-293T, HEK-293S, HEK-293F, L293, NIH-3T3, W138, BT483, Hs578T, HTB2, BT20, T47D, NS0, CRL7030, HsS78Bst cells, PER.C6, SP2, SP0, hybridoma, MRC-5, MDCK, WI-98, CAP, EB66, HUVEC, AGE1.CR, CR, cells of Trichoplusia ni, cells of Spodoptera Frugiperda, SF9, SF21, Hi5, primary T cells, Jurkat cells, mesenchymal stem cells, endothelial cells, induced pluripotent stem cells, primary cells, cells of Nicotiana tabacum, BY2, cells of Nicotiana benthamiana, cells of Oriza sativa, cells of Arabidopis thaliana, and cells of Daucus carota.

Cells for use in the present disclosure do not include cells that involve the destruction of a human embryo. Accordingly, such cells are disclaimed from and do not form part of the present disclosure.

Herein, the recombinant viral vector may be a viral vector, virus, viral-like particles (VLPs) or any combination of these, or assemblies derived from these, or assemblies derived from these and associated with a small molecule of a size less than 1000 kDa. The cell density of the cell suspension in the process of the present disclosure can be obtained by adjusting the cell density of the culture suspension by concentration and/or cultivation as described elsewhere herein.

In some embodiments, the cell density of cell suspension is adjusted by cell concentration. Any suitable concentration method known in the art can be used, such as centrifugation, filtration, microfiltration, sedimentation, acoustic settling, or specific binding, or adjusted by dilution of the cells.

In some embodiments, the cell density of cell suspension is reached by cultivation. Any suitable cultivation method known in the art can be used, such as batch cultivation, fed-batch cultivation, perfusion cultivation, chemostat, or combination of at least two of these cultivation modes, and cultivation can be performed in combination with cell concentration or in combination with cell dilution.

The transfection is performed on the day that the cell density in the cultivation reaches at least the target cell density. In the case of higher than target cell density, the total cell number needed for the transfection is calculated and the cell suspension that gives the total cell number needed is removed and diluted with fresh growth medium to reach the target cell density and target cell suspension volume. Excessive cells are then discarded from the system. After the transfection the transfected cell suspension is then transferred into a culture vessel, which can be the same as used for the cell expansion, and the production phase is carried out.

The transient transfection according to the present disclosure is a non-viral gene delivery method, either a chemical transfection method or physical transfection method. In the case of chemical transfection, it utilizes, or may use, chemical reagents selected from the group consisting of: cationic polymers such as DEAE-dextran, polybrene, polyethylenimine and derivatives thereof, dendrimers, calcium phosphate, polycations; cationic lipids including liposomal and non-liposomal transfection reagents, lipid nanoparticles.

In the case of physical transfection method, it refers to, or may refer to, one or more methods selected from the group consisting of: microinjection, optical transfection, biolistic transfection (also known as particle bombardment), electroporation, iron oxide nanoparticle delivery (also known as magnetofection), sonoporation, laser irradiation, electric field-induced molecular vibration, but is not limited thereto.

In one embodiment, the chemical reagent is a cationic polymer. A representative cationic polymer is polyethylenimine having a linear or branched topology. Typically, the polyethylenimine has a molecular weight ranging from 1 kDa to 1 000 kDa.

The transient transfection according to the process of the present disclosure is, or may be, a physical transfection method selected from the group consisting of: direct microinjection into cells or nuclei, electroporation, biolistic particle delivery (also known as particle bombardment), iron oxide nanoparticle delivery (also known as magnetofection), sonoporation, laser irradiation, electric field-induced molecular vibration, but is not limited thereto.

Microinjection is a technique that directly delivers DNA to the cell nucleus, while electroporation utilizes electrical potentials to induce the formation of pores in the cell membrane that allows the foreign DNA to enter the cells. Biolistic particle delivery or micro-projectile bombardment is a technique by which foreign genes are delivered to cells using heavy metal particles coated with exogenous DNA. Sonoporation utilizes physical disturbances in the fluid to induce pores in the cell membrane for nucleic acid delivery. Magnetofection is a transfection method that uses magnetic fields to concentrate particles containing exogenous DNA to target cells. Laser irradiation perforates individual cells by focusing a laser beam on a localized area of the cell membrane to enable the entry of nucleic acids.

In one embodiment, the transient transfection is a physical transfection method selected from the group consisting of: microinjection, optical transfection, biolistic transfection (also known as particle bombardment), electroporation, iron oxide nanoparticle delivery (also known as magnetofection).

The invention will now be illustrated by way of the following experimental section describing particular embodiments. The embodiments are meant as illustrations of the invention and are not intended to restrict the scope of the invention to the described embodiments.

### EXPERIMENTAL SECTION

### Materials and Methods

### Cell Line and Media

HEK293F cells were obtained from Thermofisher Scientific. The culture media were BalanCD HEK293 (−) glucose medium (FUJIFILM, USA) supplemented with glucose and L-glutamine, FreeStyle 293 Expression medium (Gibco), FreeStyle F17 Expression Medium (Gibco) and Viral Production Medium (Gibco) used for some transfection experiments as specified in the text. When expanded in Erlenmeyer flasks and cultured in spintubes (50 ml Corning) or TubeSpin bioreactor (50 mL TPP), cells were placed in incubator at 37° C., 5% CO2 with agitation of 120 rpm and 320 rpm respectively.

### Transfection Materials

### Chemical Transfection

Transient transfections in this study were performed via PEI mediated chemical transfection where PEI MAX (mW 40,000, Polysciences) high potency linear PEI was used. For serotype AAV1 production, the plasmid were rAAV helper-free packaging system containing 3 plasmids: pHelper, pRC1 and pGFP (Cell Biolabs). For AAV9 production, pR2C9 (pAAV2/9n Addgene) was used together with the same pHelper and pGFP used for AAV1 production. The model gene of interest GFP was used to monitor the transfection efficiency, and in transduction assay to evaluate infectious titers of the produced rAAV. HEK293F cells were grown in suspension in Erlenmeyer cell culture flasks in an incubator at 37° C. and 5% CO₂.

### Physical Transfection

Electroporator Maxcyte EXPERT STx was used together with multiple CL2 units with a processing capacity of 100 mL/unit high density cell suspension.

### Experiment Set-Up

Small scale systems were first established where key process parameters were screened and optimized. The small-scale studies were performed in mini bioreactors, i.e., 50 mL tube vessels with a vent cap, called as well spin tubes, of working volume 5 mL, and shaking in a 37° C. CO₂ incubator. These were operated in pseudo-perfusion mode, where the medium was manually exchanged by centrifugation and retention of all the cells. The developed processes were thereafter applied in perfusion cultures in bioreactor system DASGIP (Eppendorf), with a working volume of 200 mL, using alternating flow filter as cell retention device with microfilter hollow fiber cartridge (all Repligen). After transfection, the transfected cell suspensions were maintained in the same bioreactors until harvest. Culture harvest was done at 72 hours post-transfection (hpT) unless other specified in the text, by centrifugation at 200×g for 5 min and both supernatant and cell pellet were stored at −80° C. for analyses. To determine the production titers, cell pellet samples were resuspended in lysis buffer containing 50 mM Tris, 150 mM NaCl and 2 mM MgCl₂ and the cell suspension was then subjected to 3 rounds (10 min each) of freeze/thaw by alternating the tubes between isopropanol bath at −80° C. and 37° C. water bath. The crude lysates were then incubated at 37° C. with 50 Units/mL Denarase for 45 min, followed by centrifugation at 4° C. at 3000 g for 10 min to collect the clarified supernatant to proceed to further analytical assays.

### Analytical Methods

The cell density was measured by Bioprofile FLEX (Nova Biomedical) or Norma XS (Iprasense). The concentrations of glucose, lactate, glutamine, and ammonium were measured by Cedex Bio (Roche). The Transfection efficiency (GFP intensity) was measured via two flow cytometers, Gallios (Beckman Coulter) and Guava EasyCyte (Luminex Corporation). The produced viral vector capsids were quantified with the AAV1 and the AAV9 titration ELISA kits (Progen Biotechnik). The viral genome was analyzed via AAVpro titration kit for qPCR (Takara Bio) and measured using CFX96 real-time qPCR detection system (Bio-rad). Transduction assays were used to evaluate the functional infectious viral vector production. To perform the transduction assay, the supernatant or the cell lysate were added to healthy HEK293F cell suspension and GFP expression level was monitored 72 hours post transduction.

### Example 1 (Embodiment 1)

In Embodiment 1, the transient transfection process is a cationic polymer PEI (Polyethyleneimide)-mediated high cell density transfection process at 50 MVC/mL for the production of rAAV. The purpose of this experiment is to demonstrate that the high cell density transfection process is a generic process that can be applied to various transient transfection conditions of different ratios of plasmid DNA to cell number, as well as different ratios of plasmid DNA to cationic polymer.

The transfection is done via an AAV Helper free multiplasmid transient transfection system, which is a co-transfection of 3 plasmids: plasmid pAAV-RC1 (serotype AAV1), plasmid pHelper, and plasmid pGFP. The plasmid pAAV-RC1 provides the viral rep (replication) and cap (capsid) genes. The plasmid pHelper carries the adenovirus gene products required for the production of infective AAV. This function is needed to generate infective AAV particles, is provided in the body by a helper virus such as adenovirus and replaced in manufacturing by the pHelper gene only. The plasmid pGFP contains the gene coding for GFP (Green Fluorescent Protein), used as model of the gene of interest or cargo for the gene therapy. GFP is commonly used as a model gene of interest and is easily quantified thanks to fluorescence detection, demonstrating the efficiency of transfection.

HEK293 cells grown in suspension are maintained in Erlenmeyer cell culture flasks in a 37° C. CO₂ incubator. The cells at their exponential growth phase are centrifuged to reach viable cell density 50 MVC/mL and the high-density cell cultures are continued in mini bioreactors, i.e., centrifuge tubes with vent caps, volume 50 mL, in a 37° C. CO₂ incubator ready for transfection.

Several groups of transfection reagents are prepared, with varied ratios between plasmid DNA and PEI, as well as different ratios of plasmid DNA and cell number. Two groups of transfections at 1 MVC/mL, performed according to a standard transfection protocol commonly used in the field at normal cell density, are also included as references. During the transfection process, the plasmid DNAs are mixed with PEI and incubated in room temperature for 15 minutes. After the incubation, each of the transfection mixtures is added into the cell suspensions individually. No additional dilution step is performed and the cultures are maintained by pseudo-perfusion operation in the same culture vessels in an incubator until harvest at 72 hours post-transfection (hpT).

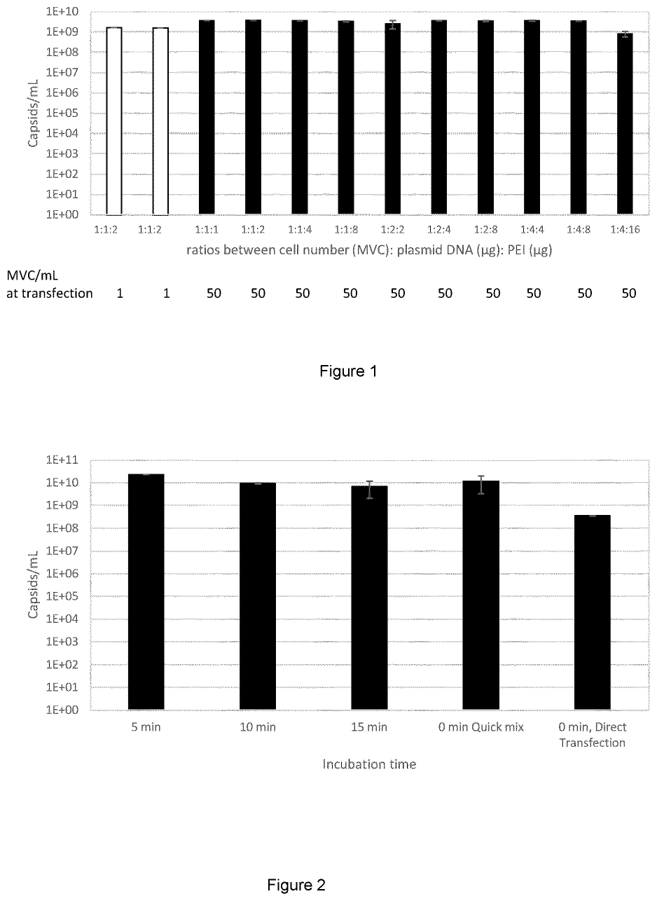

The production titers are then quantified via ELISA (Enzyme-Linked Immunosorbent Assay) that analyzes the assembled rAAV capsids in the harvest. In FIG. 1 the two groups counting from the left are controls, where the cell densities at transfection were 1 MVC/mL. From the 3rd group onwards, the transfections were done at 50 MVC/mL with various ratios between cells, plasmid DNA and PEI. The groups are named after ratios between cell number (MVC): plasmid DNA (μg): PEI (μg): In the groups 1:1:1, 1:1:2, 1:1:4 and 1:1:8, the ratio of plasmid DNA to cell number is 1 μg DNA per 1 million cells, with DNA (μg): PEI (μg) ratios 1:1, 1:2, 1:4 and 1:8 respectively. In the groups 1:2:2, 1:2:4 and 1:2:8, the plasmid DNA is 2 μg DNA per 1 million cells, with DNA (μg): PEI (μg) ratios 1:1, 1:2 and 1:4 respectively. In the groups 1:4:4, 1:4:8 and 1:4:16, the plasmids DNA is 4 μg DNA per 1 million cells, with plasmid DNA (μg): PEI (μg) ratios 1:1, 1:2 and 1:4 respectively. It can be seen from the figure that except the last group, 1 million cells: 4 μg plasmid DNA: 16 μg PEI, all the other groups give higher production titers compared to the control groups performed at 1 MVC/mL. The cytotoxicity of PEI is well known due to its cationic character; therefore it is reasonable to see a decrease of productivity when the transfection reagents are over used.

### Example 2 (Embodiment 2)

In Embodiment 2, the transient transfection process is a cationic polymer PEI-mediated high cell density transfection process at 50 MVC/mL for the production of rAAV. The purpose of this experiment is to demonstrate the very high cell density transfection process with different incubation times of the transfection reagent that is a mix of the plasmid DNAs and cationic polymer. The transfection is done via an AAV Helper free multiplasmid transient transfection system, which is a co-transfection of 3 plasmids: pAAV-RC1, pHelper, and pGFP. The plasmid pAAV-RC1 provides the viral rep (replication) and cap (capsid) genes. The plasmid pHelper carries the adenovirus gene products required for the production of infective AAV. The plasmid pHelper carries the adenovirus gene products required for the production of infective AAV. This function is needed to generate infective AAV particles, is provided in the body by a helper virus such as adenovirus and replaced in manufacturing by the pHelper gene only. The plasmid pGFP contains the gene coding for GFP (Green Fluorescent Protein), used as model of the gene of interest or cargo for the gene therapy. GFP is commonly used as a model gene of interest and is easily quantified thanks to fluorescence detection, demonstrating the efficiency of transfection.

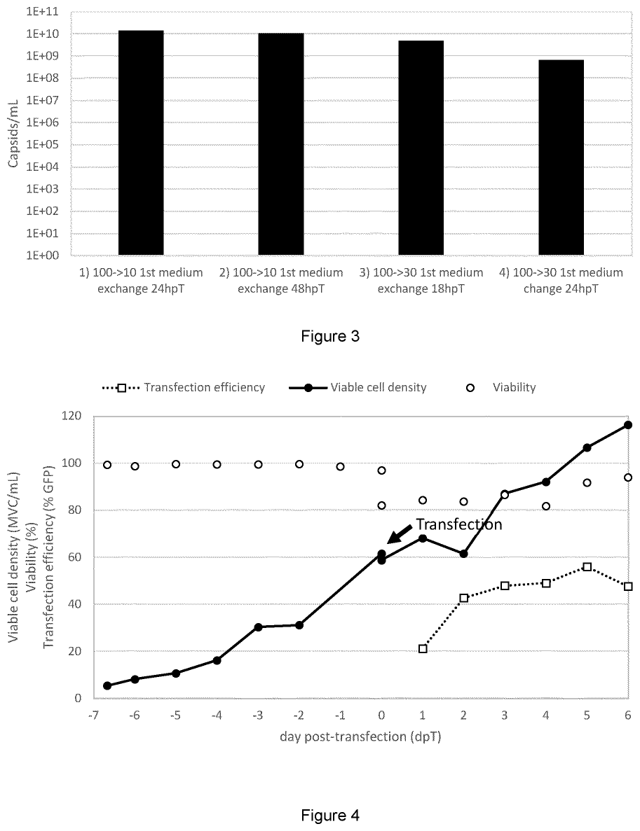

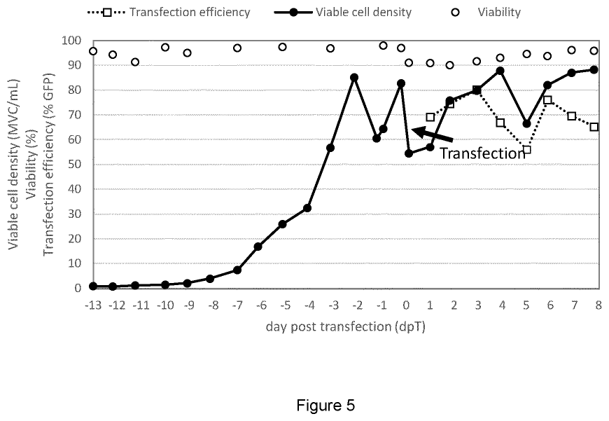

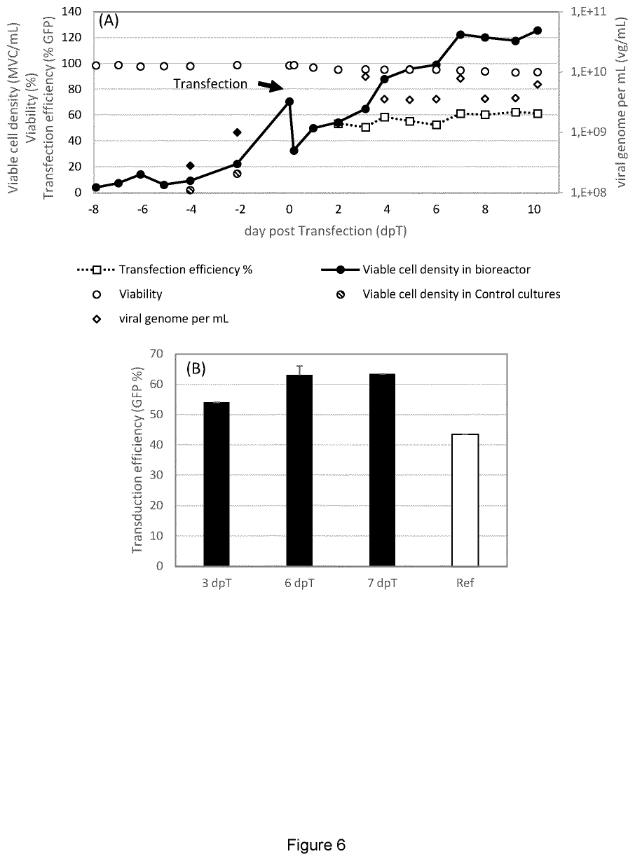

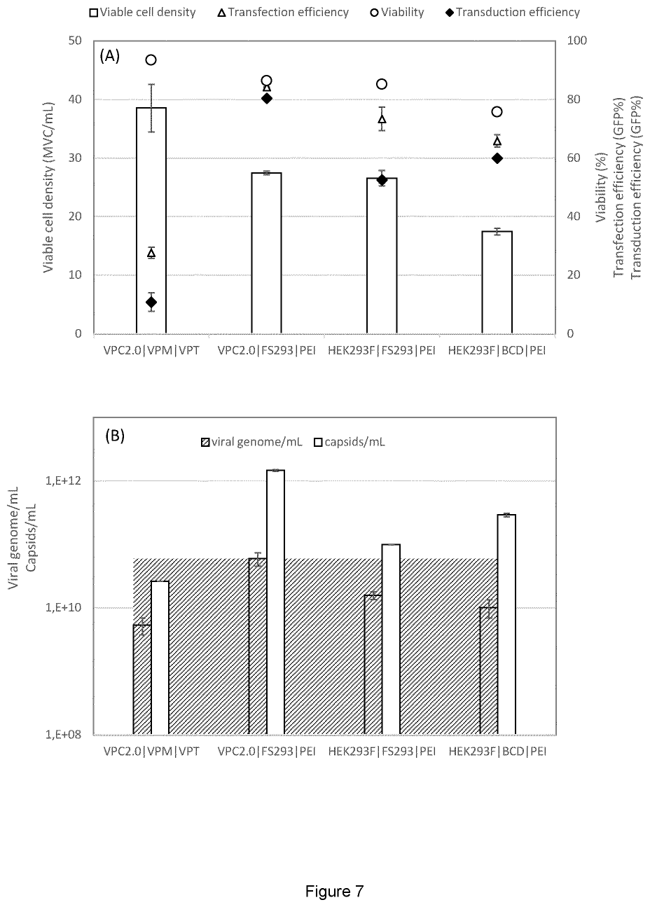

HEK293 cells grown in suspension are maintained in Erlenmeyer cell culture flasks in a 37° C. CO₂ incubator. The cells at their exponential growth phase are centrifuged to reach viable cell density 50 MVC/mL and the high density cell cultures are continued in mini bioreactors in pseudo-perfusion mode, i.e., volume 50 mL vessels with vent caps, in a 37° C. CO₂ incubator ready for transfection. The plasmid DNA is 1 μg per 1 million cells, and the DNA (μg): PEI (μg) ratio is 1:2 in all the groups. Using such ratios, the usage of both plasmid DNA and PEI are the lowest with equivalent production titer from described in embodiment 1, which clearly reduces the costs associated to the plasmid production and to the PEI. After mixing DNA and PEI, different incubation times are used: 5 minutes ‘5 min’, 10 minutes ‘10 min’ or 15 minutes ‘15 min’ are applied (see FIG. 2). After the incubation, the mixture of DNA and PEI is added to the cell suspension. In the group ‘0 min Quick mix’, the DNA is mixed with PEI and a quick mix is done via inverting the mixture a few times before adding immediately into the cell suspension without any incubation. In the group ‘0 min, Direct transfection’, the DNA is added into the cell suspension, followed by immediate addition of PEI into the cell suspension, and the pre-mixing of plasmid DNA and PEI is completely eliminated. In all the groups, no further dilution step than 3:5 brought by adding 2 mL of mix DNA and PEI to 3 mL cell suspension at 50 MVC/mL to carry out the transfection, is performed after the transfection and the cultures are maintained in the same culture vessels in an incubator until harvest at 72 hpT. The production titers are then quantified via ELISA that analyzes the assembled rAAV capsids in the harvest. FIG. 2 shows that in the final harvest, all the used procedures ensure the production of AAV capsids, with productions comparable in all the groups except for ‘0 min, Direct transfection’ giving a lower AAV production. This demonstrates that the transfection can be performed with or without the formation of the cationic polymer/DNA complex, and different incubation times are applicable in case of a pre-mixing of cationic polymer and DNA.

### Example 3 (Embodiment 3)

In Embodiment 3, the transient transfection process is a PEI-mediated high cell density transfection process at 100 MVC/mL for the production of rAAV. The purpose of this experiment is to demonstrate the extremely high cell density transfection process with different dilutions to various high cell densities (10 MVC/mL as control and 30 MVC/mL) after the transfection into the production phase. The transfection is done via an AAV Helper free multiplasmid transient transfection system, which is a co-transfection of 3 plasmids: pAAV-RC1, pHelper, and pGFP. The plasmid pHelper carries the adenovirus gene products required for the production of infective AAV. This function is needed to generate infective AAV particles, is provided in the body by a helper virus such as adenovirus, and replaced in manufacturing by the pHelper gene only. The plasmid pGFP contains the gene coding for GFP (Green Fluorescent Protein), used as model of the gene of interest or cargo for the gene therapy. GFP is commonly used as a model gene of interest and is easily quantified thanks to fluorescence detection, demonstrating the efficiency of transfection.

HEK293 cells grown in suspension are maintained in Erlenmeyer cell culture flasks in a 37° C. CO₂ incubator. Before the transfection, cells in exponential growth phase are centrifuged to reach a viable cell density of 100 MVC/mL. The amount of plasmid DNA used is 1 μg per 1 million cells, and the plasmid DNA (μg): PEI (μg) ratio is 1:2 in all the groups. Using such ratios, the usage of both plasmid DNA and PEI are the lowest with equivalent production titer from described in embodiment 1. The transfection process is exactly the same for all the groups: plasmid DNAs are mixed with PEI and incubated for 15 minutes before added into the extremely high-density cell suspension. After the transfection the transfected cell suspensions are diluted into 4 different conditions for the production phase, two of them to 10 MVC/mL and two to 30 MVC/mL. Before the final harvest at 72 hpT, different medium exchange strategies of pseudo-perfusion are applied in each condition:
1) a first condition with dilution to 10 MVC/mL, and medium exchange performed at 24 hours post-transfection (hpT) and once 24 hours after that;
2) a second condition with dilution to 10 MVC/mL, and one medium exchange performed at 48 hpT;
3) a third condition with dilution to 30 MVC/mL, and medium exchange performed at 18 hpT and once per 24 hours after that;
4) a fourth condition with dilution to 30 MVC/mL, and medium exchange performed at 24 hpT and once per 24 hours after that.

After dilution the cultures are continued in mini bioreactors, i.e., volume 50 ml tube vessels with vent caps, in a 37° C. CO₂ incubator until harvest at 72 hpT. The production titers are then quantified via ELISA that analyzes the assembled rAAV capsids in the harvest. FIG. 3 indicates that in a simple culture system such as a 50 ml tube vessels with a vent cap shaking in a 37° C. CO₂ incubator during the production phase, transfections at extremely high cell density 100 MVC/mL produce comparable titers after dilution to cell densities 10 or 30 MVC/mL, as long as medium exchange is performed to replenish the nutrients and remove the byproducts to maintain the health of the cell culture, i.e. condition 3 in comparison with conditions 1 and 2. In conditions 4, the medium exchange is first performed at 24 hpT, which leads to a lower rAAV production in comparison to condition 3 with a first medium exchange at 18 hours. This finding implies that the dilution after transfection is not a requirement in case that the cell culture can be maintained at very high density, such as 100 MVC/mL, with sufficient medium exchange.

### Example 4 (Embodiment 4)

In Embodiment 4, the transient transfection process is a PEI-mediated very high cell density transfection process at 60 MVC/mL for the production of rAAV in a perfusion bioreactor culture with 220 mL working volume. The purpose of this experiment is to demonstrate the very high cell density transfection process in a perfusion bioreactor culture without any dilution after the transfection for production of rAAV. The transfection is done via an AAV Helper free multiplasmid transient transfection system, which is a co-transfection of 3 plasmids: pAAV-RC1, pHelper, and pGFP. The plasmid pHelper carries the adenovirus gene products required for the production of infective AAV. This function is needed to generate infective AAV particles, is provided in the body by a helper virus such as adenovirus and replaced in manufacturing by the pHelper gene only. The plasmid pGFP contains the gene coding for GFP (Green Fluorescent Protein), used as model of the gene of interest or cargo for the gene therapy. GFP is commonly used as a model gene of interest and is easily quantified thanks to fluorescence detection, demonstrating the efficiency of transfection.

HEK293 cells grown in suspension are inoculated 7 days prior to the transfection in a bioreactor with 220 mL working volume and perfusion is started shortly after inoculation using hollow fiber cartridge operating in ATF (Alternating Tangential Flow) mode. The transfection is targeted at a cell density between 60 and 100 MVC/mL. On the day that the viable cell density reaches 61 MVC/mL, the transfection reagents are prepared with the ratios presented in embodiment 2 & 3: Plasmid DNA 1 μg per 1 million cells, and DNA (μg): PEI (μg) ratio 1:2. Using such ratios, the usage of both plasmid DNA and PEI are the lowest with equivalent production titer as presented in embodiment 1, which clearly reduces the production cost, especially in bioreactor scales. The transfection efficiency is monitored daily using a flow cytometer that quantifies the GFP expression level. Here the culture is harvested when no further increase in the GFP level is observed. The results are presented in FIG. 4. A drop in viable cell density after transfection is occurring from 61.4 to 58.7 MVC/mL due to the added volume from transfection reagents. There is a decline in viability from 96% measured just before the transfection down to 82% at measured 4 hpT (0 dpT), which is a normal viability decrease as transfection response. A reduction of the cell growth is also noted during the first 2 days post-transfection. The slowly recovering cell growth and viability after 2 dpT are indication that the plasmids are gradually lost during the cell division, which is a normal phenomenon in such transient transfection. The transfection efficiency increases daily after transfection and is maximal at 56% at 5 dpT, then slightly decreasing at 6 dpT. The culture is harvested at 6 dpT. At that time, the cell density and viability are restored to 116 MVC/mL and 94% respectively.

### Example 5 (Embodiment 5)

In Embodiment 5, the transient transfection process is a flow electroporation process at >80 MVC/mL for the production of rAAV in a perfusion bioreactor culture with 200 mL working volume. The purpose of this experiment is to demonstrate the extremely high cell density transfection process in a perfusion bioreactor culture without any dilution after the transfection for production of rAAV. The transfection is done via an AAV Helper free multiplasmid transient transfection system, which is a co-transfection of 3 plasmids: pAAV-RC1, pHelper, and pGFP. The plasmid pHelper carries the adenovirus gene products required for the production of infective AAV. This function is needed to generate infective AAV particles, is provided in the body by a helper virus such as adenovirus and replaced in manufacturing by the pHelper gene only. The plasmid pGFP contains the gene coding for GFP (Green Fluorescent Protein), used as model of the gene of interest or cargo for the gene therapy. GFP is commonly used as a model gene of interest and is easily quantified thanks to fluorescence detection, demonstrating the efficiency of transfection.

HEK293 cells grown in suspension are inoculated 13 days prior to the transfection in a bioreactor with 200 mL working volume and perfusion is started after cell density reaches 2 MVC/mL, using hollow fiber cartridge operating in ATF mode. The electroporator EXPERT STx from Maxcyte is used here. In normal function of this system according to the manufacturer and currently used in the field, cells proliferated in culture at low cell density around 1 to 2 MVC/mL are concentrated by centrifugation to a concentration of 100 MVC/mL and transferred to the “sample bag” of the Maxcyte system. The electroporation takes place as flow electroporation and the resulting material is collected in the “collection bag”.

In the present disclosure, before the experiment, the electroporator EXPERT STx from Maxcyte is remodeled with both the sample bag and collection bag removed and the electroporation unit on STx is connected directly “in-line” with the bioreactor A function as the “sample bag” and a collection vessel B function as the “collection bag” of Maxcyte. The collection vessel B is connected to bioreactor A as well, to transfer the collected cell suspension of collection vessel B back to bioreactor A. The collection vessel B is an inflatable bag of 10 L, i.e., a bag typically used for storage of culture medium or buffer, and is pre-inflated with sterile air containing 5% carbon dioxide. The transfection is targeted at a cell density that is between 80 and 100 MVC/mL. On the day that the viable cell density reaches 83 MVC/mL, electroporation with 80 mg plasmid DNA is carried out, i.e. 400 μg plasmid DNA per mL of culture. This electroporation is done directly on the cell suspension at 80 MVC/mL from bioreactor A using two electroporation units CL2. Each CL2 unit has a processing capacity of 100 mL high density cell suspension, therefore two CL2 units are sequentially used for the electroporation of 200 mL cell suspension. The cell suspension from bioreactor A is pumped into the electroporation unit, electroporated, and pumped to the collection vessel B with 3 mL per cycle until the whole 200 mL cell suspension has been electroporated. 10 mL of DNase1 are added to the collection vessel B to neutralize excessive free DNA in the electroporated cell suspension. After electroporation, the cells are transferred to collection vessel B, which is then laid down in a 37° C. incubator for 45 minutes. In this way the transfected cells are well distributed on a large enough surface with access to oxygen for the recovery. After 45 minutes of recovery time period, the transfected cell suspension is then transferred back into bioreactor A. From the moment that the transfected cells are transferred back to bioreactor A, the cell culture enters the production phase and the transfection efficiency is monitored daily using a flow cytometer that quantifies the GFP expression level. FIG. 5 shows the results. A loss in viable cell density occurs immediately after transfection. This is due to the sudden cell death caused by electroporation technique. There is a decline in viability from 97% to 91% after the transfection, which is a normal transfection response for electroporation technique.

The GFP signal peaks at a maximum of 80% at 3 days post transfection and gradually decreases afterward. The culture is harvested when severe decline of viable cell density, viability and GFP signal are observed, which is day 8 post transfection in the present case. Note the cell density decrease two days before the transfection is due to manual removal of part of the culture to control the cell density in the target range preparing for the transfection.

### Example 6 (Embodiment 6)

In Embodiment 6, the transient transfection process is a PEI-mediated very high cell density transfection process at 50 MVC/mL for the production of rAAV in a perfusion bioreactor culture with 200 mL working volume. The purpose of this experiment is to demonstrate the very high cell density transfection process in a perfusion bioreactor culture without any dilution after the transfection for production of rAAV. The transfection is done via an AAV Helper free multiplasmid transient transfection system, which is a co-transfection of 3 plasmids: pAAV-RC9, pHelper, and pGFP. Plasmid pAAV-RC9 provides the viral rep (replication) and cap (capsid) genes. The plasmid pHelper carries the adenovirus gene products required for the production of infective AAV. This function is needed to generate infective AAV particles, is provided in the body by a helper virus such as adenovirus and replaced in manufacturing by the pHelper gene only. The plasmid pGFP contains the gene coding for GFP (Green Fluorescent Protein), used as model of the gene of interest or cargo for the gene therapy. GFP is commonly used as a model gene of interest and is easily quantified thanks to fluorescence detection, demonstrating the efficiency of transfection. HEK293 cells grown in suspension are inoculated 8 days prior to the transfection in a bioreactor A with 200 mL working volume and perfusion is started shortly after inoculation using hollow fiber cartridge operating in ATF (Alternating Tangential Flow) mode. The transfection is targeted at a cell density between 50 and 80 MVC/mL. On the day that the viable cell density reaches 70 MVC/mL, 120 mL of cell suspension at 50 MVC/mL are prepared by mixing 85 mL of suspension with 35 mL fresh culture medium. The transfection reagents are prepared with the ratios: Plasmid DNA 0.5 μg per 1 million cells, and DNA (μg): PEI (μg) ratio 1:2. Using such ratios, the usage of both plasmid DNA and PEI are substantially decreased, which clearly reduces the production cost, especially in bioreactor scales. A total of 30 mg of plasmid DNA composed of pRC9: pGFP:pHelper with ratio 1:1:2 is diluted with culture medium to a total of 40 mL. The total 60 mg of PEI is also diluted with culture medium to a volume of 40 mL. The 40 mL plasmid DNA and 40 mL PEI solution are mixed, generating the transfection reagent mix, and submitted to 15 minutes incubation of in room temperature. This transfection reagent mix is then added into 120 mL of cell suspension at 50 mVC/mL. After mixing this cell suspension and the transfection reagent mix, the transfected cell suspension is transferred into a tissue culture flask with 300 cm² surface area, on non-tissue culture treated surface, for 30 minutes. After the 30 minutes cell recovery time period, the transfected cell suspension is transferred back into the bioreactor. From the moment that the transfected cells are transferred back to the bioreactor, the cell culture enters the production phase and the transfection efficiency is monitored daily using a flow cytometer that quantifies the GFP expression level. Here the culture is harvested when no further increase in the GFP level is observed, which is 10 days post transfection. Daily cell suspension samples are collected from the bioreactor A during the production phase. The supernatant from samples obtained by centrifugation is used to perform transduction assays. Transduction assays are used to evaluate how functional the infectious viral vectors contained in the sample are, by putting the sample in presence of cells susceptible to be infected by these infectious viral vectors. To perform the transduction assay, the supernatant of collected samples are added into healthy HEK293F cell suspension and GFP expression level is monitored up to 72 hours post transduction or 3 days post transduction. FIG. 6A shows the viable cell density, viability, transfection efficiency and production titers. It demonstrates a long-term continuous production phase that is very long and has very high cell density, i.e., 120 MVC/mL maintained for 4 days since 7 dpT. For comparison, two control cultures in mini bioreactor with transfection performed at 1 MVC/mL cell density (left pattern-filled circle) with viral genome production/mL (left filled diamond) and with transfection performed at 15 MVC/mL cell density (right pattern-filled circle) with viral genome production/mL (right filled diamond), are represented in the same graph for convenience but the time scale does not apply for these. The perfusion process generated 33-fold higher cell density and 30-fold higher vg/mL to the 1 MVC/mL control culture that was harvested at 2 MVC/mL on its 3 dpT. The perfusion process generated 4.3-fold higher cell density and 8.4-fold higher vg/mL comparing to the 15 MVC/mL small scale process. It can be concluded that the perfusion process maintained the cell specific productivity very well comparing to the 1 MVC/mL standard transfection process, and almost doubled the cell specific productivity when comparing to 15 MVC/mL processes in spin tubes. This is potentially due to the continuous medium renewal in a perfusion culture which is not feasible in batch-mode small scale systems. FIG. 6B shows the results of transduction assay for the same process. The groups 3 dpT, 6 dpT and 7 dpT refer to supernatant that is collected 3 days post transfection, 6 days post transfection and 7 days post transfection, respectively. The Ref group is the reference group in which the production of rAAV by transfection is done in a shake flask at low cell density 1 MVC/mL and supernatant is collected 3 days post transfection, which is the common practice for rAAV production. It demonstrates a long-term continuous production phase of functional rAAV vectors that are infectious to human cells, which gives about 50% increase in the infectious titer comparing to the reference group that is transfected at 1 MVC/mL using the same cells and transfection reagents. This embodiment has demonstrated the possibility of reaching very high titer of functional rAAV vectors with a prolonged production phase.

### Example 7

In Embodiment 7, the transient transfection processes were performed at 50 MVC/mL for the production of rAAV using both host cells, HEK293F and Viral production cells 2.0 (VPC2.0, a variant cell line of HEK293 cells obtained from ThermoFisher). Two different chemical transfection methods: cationic polymer PEI and cationic lipid-based transfection reagent (VPT) were used and three transfection media: Viral production medium (VPM), FreeStyle293 (FS293) and BalanCD HEK (BCD) were used. The purpose of this experiment is to demonstrate that the current invention is applicable to various producer cell lines, chemical transfection methods, and transfection media that are commercially available and widely adopted in the field. The transfection was done via an AAV Helper free multiplasmid transient transfection system, which was a co-transfection of 3 plasmids: pAAV-RC9, pHelper, and pGFP. The plasmid pAAV-RC9 provides the viral rep (replication) and cap (capsid) genes. The plasmid pHelper carries the adenovirus gene products required for the production of infective AAV. The plasmid pHelper carries the adenovirus gene products required for the production of infective AAV. This function is needed to generate infective AAV particles, is provided in the body by a helper virus such as adenovirus, and replaced in manufacturing by the pHelper gene only. The plasmid pGFP contains the gene coding for GFP (Green Fluorescent Protein), used as model of the gene of interest or cargo for the gene therapy. GFP is commonly used as a model gene of interest and is easily quantified thanks to fluorescence detection, demonstrating the efficiency of transfection.

HEK293F cells and VPC2.0 cells grown in suspension were maintained in Erlenmeyer cell culture flasks in a 37° C. CO₂ incubator. The cells at their exponential growth phase were centrifuged to reach viable cell density 50 MVC/mL and the high-density cell cultures were continued in mini bioreactors, i.e., volume 50 mL vessels with vent caps, in a 37° C. CO₂ incubator ready for transfection. The plasmid DNA was 1 μg per 1 million cells, and the DNA (μg): PEI (μg) ratio was 1:2 in all the groups. After 10-15 minutes incubation, the mixture of DNA and PEI was added to the cell suspension. In all the groups, no further dilution step was performed after the transfection and the cultures were maintained in the same culture vessels in an incubator until harvest at 72 hpT. The production titers were then obtained via ELISA to quantify the assembled rAAV capsids and qPCR to quantify the viral genome. FIG. 7 shows 4 different transfection processes: VPC2.0 cells transfected with VPT reagent in VPM, VPC2.0 cells transfected with PEI in FS293 medium, HEK293F cells transfected with PEI in FS293 medium, and HEK293F cells transfected with PEI in BCD medium (from left to right). FIG. 7A shows the cell densities, viabilities and transfection efficiency GFP % at the harvest. It also includes the results from transduction assay that was performed using the supernatant after centrifugation of the final harvested cell suspension. FIG. 7B shows the production titers of viral genome/mL and capsids/mL in different groups respectively. This demonstrates that the current invention can be applied to various transfection systems that use different producer cell lines and transfection media, even different transfection methods.

### REFERENCES

1. Xiao, et al. “Production of high-titer recombinant adeno-associated virus vectors in the absence of helper adenovirus.” J Virol 72 (1998): 2224-2232;
2. Grimm, et al. “Novel tools for production and purification of recombinant adeno associated virus vectors.” Hum Gene Ther 9 (1998): 2745-2760;
3. Matsushita, et al. “Adeno-associated virus vectors can be efficiently produced without helper virus.” Gene Ther 5 (1998): 938-945;
4. Maranga, L., et al. 2005. “Characterization of changes in PER. C6™ cellular metabolism during growth and propagation of a replication-deficient adenovirus vector.” Biotechnology and bioengineering, 90(5), pp. 645-655;
5. Henry, O. et al. 2005. “Metabolic flux analysis of HEK-293 cells in perfusion cultures for the production of adenoviral vectors.” Metabolic engineering, 7(5-6), pp. 467-476;
6. Joshi et al., 2021. “Advancements in molecular design and bioprocessing of recombinant adeno-associated virus gene delivery vectors using the insect-cell baculovirus expression platform”. Biotechnology Journal, 16(4), p. 2000021;
7. Meghrous et al., 2005. “Production of recombinant adeno-associated viral vectors using a baculovirus/insect cell suspension culture system: From shake flasks to a 20-L bioreactor”. Biotechnology progress, 21(1), pp. 154-160;
8. Urabe et al., 2002. “Insect cells as a factory to produce adeno-associated virus type 2 vectors”. Human gene therapy, 13(16), pp. 1935-1943;
9. Riedl, et al. “Non-Viral Transfection of Human T Lymphocytes.” Processes 2018, 6, 188;
10. Backliwal et al. “High-density transfection with HEK-293 cells allows doubling of transient titers and removes need for a priori DNA complex formation with PEI.” Biotechnol Bioeng. 2008 Feb. 15; 99(3):721-7;
11. Blackstock et al. “Comprehensive Flow Cytometry Analysis of PEI-Based Transfections for Virus-Like Particle Production”, Research, 2020, Article ID 1387402;
12. Steger et al. CHO-S antibody titers >1 gram/liter using flow electroporation-mediated transient gene expression followed by rapid migration to high-yield stable cell lines. J Biomol Screen. 2015 April; 20(4):545-51.

1. A process for producing recombinant vectors in eukaryotic cells, said method comprising the steps of:
a) obtaining a cell suspension having a cell density of at least 25 million viable cells per milliliter;
b) performing a transient transfection of the cells in the cell suspension with an exogenous plasmid DNA or a plurality of different exogenous plasmid DNAs to obtain transfected cells;
c) optionally performing a cell recovery for a period of time, wherein during the cell recovery period, said transfected cells are kept essentially still on a surface; and
d) following the transfection of step b), and/or the optional cell recovery step c), limited or no dilution is performed of said transfected cells present in said cell suspension and wherein production of said recombinant vectors is thereafter initiated from a culture of transfected cells having a cell density of at least 20 million viable cells per milliliter.

2. The process of claim 1, wherein said cell suspension of step a) has a cell density of at least 30 million cells per milliliter.

3. The process of claim 1, wherein said cell suspension of step a) has a cell density of at least 50 million cells per milliliter.

4. The process of claim 1, wherein said cell suspension of step a) has a cell density of at least 80 million cells per milliliter.

5. The process of claim 1, wherein said cell suspension of step a) has a cell density of at least 100 million cells per milliliter.

6. The process of claim 1, wherein said process comprises medium exchange performed at one or more points in time when the production of step d) has been initiated.

7. The process of claim 1, wherein said process comprises medium exchange carried out by perfusion when the production of step d) has been initiated.

8. The process of claim 7, wherein said perfusion is performed in a perfusion bioreactor culture.

9. The process of claim 1, wherein the production of step d) is performed for a period of at least 24 hours.

10. The process of claim 6, wherein no dilution that would generate a cell density lower than 20 MVC/mL is performed before the initiation of the production of step d).

11. The process of claim 1, wherein said cell suspension of step a) is such that the cells of said suspension occupy at least a fraction of 6 percent of a culture volume and wherein the cell suspension is selected from the group consisting of a cell culture, a microorganism fermentation, a cell suspension derived from cell tissue and a cell suspension derived from blood.

12. The process of claim 1, wherein the eukaryotic cells are selected from the group consisting of mammalian cells, human cells, avian cells, insect cells and plant cells.

13. The process of claim 12, wherein the cells are selected from the group consisting of: CHO, CHO-DBX11, CHO-DG44, CHO-S, CHO-K1, Vero, BHK, HeLa, COS, MDCK, HEK-293, HEK-293T, HEK-293S, HEK-293F, L293, NIH-3T3, W138, BT483, Hs578T, HTB2, BT20, T47D, NS0, CRL7030, HsS78Bst cells, PER.C6, SP2, SP0, hybridoma, MRC-5, MDCK, WI-98, CAP, EB66, HUVEC, AGE1.CR, CR, cells of Trichoplusia ni, cells of Spodoptera Frugiperda, SF9, SF21, Hi5, mesenchymal stem cells, endothelial cells, induced pluripotent stem cells, primary cells, cells of Nicotiana tabacum, BY2, cells of Nicotiana benthamiana, cells of Oriza sativa, cells of Arabidopis thaliana, and cells of Daucus carota.

14. The process of claim 1, wherein the recombinant viral vector is a viral vector, a virus, viral-like particles (VLPs) or any combination of these, or assemblies derived from these, or assemblies derived from these and associated with a small molecule of a size less than 1000 kDa.

15. The process of claim 1, wherein recombinant Adeno-Associated Virus (AAV) viral vector are produced, optionally wherein the transfection of step b) is performed using an (AAV) transient transfection system comprising co-transfection of at least three plasmids.

16. The process of claim 1, wherein the cell density of step a) is obtained by adjusting the cell density of the culture suspension by concentrating the content of the cells of the cell suspension and/or by cultivation of the cells in the culture suspension.

17. The process of claim 1, wherein the cell density of step a) is adjusted by concentrating the cells of the suspension.

18. The process of claim 1, wherein the cell density of step a) is adjusted by cultivation, and wherein cultivation may be performed in combination with cell concentration and/or in combination with cell dilution.

19. The process of claim 1, wherein said transient transfection comprises chemical transfections utilizing chemical reagent selected from the group consisting of: cationic polymers, dendrimers, calcium phosphate, polycations, cationic lipids transfection reagents and lipid nanoparticles.

20. The process of claim 25, wherein the said polyethylenimine is a synthesized cationic polymer with topologies of linear or branched forms, with a molecular weight ranging from 1 kDa to 1,000 kDa.

21. The process of claim 1, wherein said transfection comprises physical transfection methods from the group consisting of: microinjection, optical transection, biolistic transfection (also known as particle bombardment), electroporation, iron oxide nanoparticle delivery (also known as magnetofection), sonoporation, laser irradiation, and electric field-induced molecular vibration.

22. NEW The process of claim 9, wherein the production of step d) is performed for a period of at least 48 hours, at least 72 hours, or for 4, 5, 6, 7, 7, 9 or 10 days.

23. NEW The process of claim 17, wherein the cells are concentrated by centrifugation, filtration, microfiltration, sedimentation, acoustic settling, specific binding of the cells, or by diluting the cells of the suspension.

24. NEW The process of claim 18, wherein the cultivation is batch cultivation, fed-batch cultivation, perfusion cultivation, chemostat, or combination of at least two of these cultivation modes.

25. NEW The process of claim 19, wherein the cationic polymer is DEAE-dextran, polybrene or polyethylenimine and derivates thereof, alternatively the cationic lipid is a liposomal or non-liposomal transfection reagent.
